(19) United States
(12) Patent Application Publication
(10) Pub. No.: US 2003/0083318 A1
(43) Pub. Date: May 1, 2003

(54) Therapy for neurodegenerative diseases

(76) Inventors: Jean-Pierre Julien, Montreal (CA); Jasna Kriz, Montreal (CA)

Correspondence Address: OGILVY RENAULT, 1981 MCGILL COLLEGE AVENUE, SUITE 1600, MONTREAL, QC, H3A2Y3, CA

(21) Appl. No.: 10/279,725

(22) Filed: Oct. 25, 2002

Related U.S. Application Data
(60) Provisional application No. 60/330,599, filed on Oct. 25, 2001.

(51) Int. Cl. A61K 31/65; A61K 31/426; A61K 31/44

(52) U.S. Cl. 514/153; 514/355; 514/369

(57) ABSTRACT

The present invention relates to a method for reducing symptoms related to a neurodegenerative disease and/or treating a neurodegenerative disease, this therapy comprising the administration of a therapeutically effective amount of at least two compounds selected from the group of an inhibitor of microglial activation, an antiglutominergic agent and a voltage gated calcium channer blocker to a patient suffering from a neurodegenerative disease. The present invention also relates to a composition for reducing symptoms related to a neurodegenerative disease and/or treating a neurodegenerative disease, comprising a therapeutically effective amount of at least two compounds selected from the group of an inhibitor of microglial activation, an antiglutaminergic agent and a voltage gated calcium channel blocker in association with a pharmaceutically acceptable carrier.

### BACKGROUND OF THE INVENTION

(a) Field of the Invention

This invention relates a new therapy for the treatment of neurodegenerative diseases, this new therapy delaying the onset of the disease and increasing the survival time of the subject.

(b) Description of Prior Art

Amyotrophic lateral sclerosis (ALS) is a late onset neurodegenerative disease characterized by progressive muscle weakness, muscle atrophy and eventual paralysis, leading to death within 2-5 years (Julien, J.-P. (2001) Cell 104, 581-591; Rowland, L. P. & Shneider, N. A. (2001) N. Eng. J. Med. 344, 1688-1699). The disease occurs in both sporadic and familial forms with highly similar clinical courses. Familial forms of ALS (FALS), being inherited in an autosomal dominant pattern, make up ˜10% of all ALS cases. In 20% of FALS, missense mutations have identified in the gene coding for superoxide dismutase 1 (SOD1). Transgenic mice overexpressing SOD1 mutants linked to FALS develop progressive motor neuron disease with many pathological features observed in both familial and sporadic ALS cases (Julien, J.-P. (2001) Cell 104, 581-591; Rowland, L. P. & Shneider, N. A. (2001) N. Eng. J. Med. 344, 1688-1699; Wong, P. C., et al. (1995) Neuron 14, 1105-1116). Yet, the mechanism of SOD1-mediated, as well as the sporadic form of disease is not fully understood. The current view is that the motor neuron death in ALS is complex and may involve multiple pathways including formation of protein aggregates, proteosome dysfunction, axonal transport defects, oxidative damage, mitochondrial defects, alterations in calcium homeostasis, caspase activation, and changes in levels of Bcl-2 members. In addition, excitotoxicity due to astrocyte dysfunction, impaired glutamate transport or altered glutamate receptor function, and inflammatory processes from microglia activation are other factors likely to be involved in the propagation of the neurodegenerative process (Rothstein, J. D., et al. (1995) Ann. Neurol. 38, 73-84; Almer, G., et al. (1999) J. Neurochem.72, 2415-2425).

At the present, there is no effective pharmacological treatment for ALS. The only currently approved therapy, the antiglutaminergic agent riluzole, has been shown to have only a marginal survival benefit (Julien, J.-P. (2001) Cell 104, 581-591; Rowland, L. P. & Shneider, N. A. (2001) N. Eng. J. Med. 344, 1688-1699). In two clinical trials, riluzole prolonged survival by three to six months (Rowland, L. P. & Shneider, N. A. (2001) N. Eng. J. Med. 344, 1688-1699). When tested in transgenic animals with mutant SOD1, riluzole extended survival by 13-15 days with no significant effects on the onset of disease (Gurney, M. E., et al. (1996) Ann. Neurol. 39, 147-157).

Recently, several reports showed that minocycline, a second-generation tetracycline, can exhibit biological effects completely distinct from its antimicrobial action (Yrjänheikki, J., et al. (1998) Proc. Natl. Acad. Sci. USA 95, 15769-15774; Yrjänheikki, J., et al. (1999) Proc. Natl. Acad. Sci. USA 96, 13496-13500; Chen, M., et al. (2000) Nat. Med. 6, 797-801). Thus, in experimental model of cerebral ischemia, minocycline inhibited microglial activation, reduced inflammation and decreased the size of infarct (Yrjänheikki, J., et al. (1998) Proc. Natl. Acad. Sci. USA 95, 15769-15774). It also inhibits caspase-1 and inducible nitric oxide synthase (iNOS) upregulation (Yrjänheikki, J., et al. (1998) Proc. Natl. Acad. Sci. USA 95, 15769-15774; Yrjänheikki, J., et al. (1999) Proc. Natl. Acad. Sci. USA 96, 13496-13500; Chen, M., et al. (2000) Nat. Med. 6, 797-801). Moreover, minocycline was able to delay mortality and to inhibit caspase-1 and -3 upregulation in a mouse model of Huntington disease (Chen, M., et al. (2000) Nat. Med. 6, 797-801). In addition, results of our recent study demonstrated that minocyline inhibits microglial activation and delays mortality in SOD1 mutant mice.

Numerous studies have suggested that increased intracellular calcium is associated with motor neuron injury. Motor neurons from ALS patients may contain elevated calcium levels due to increased mitochondrial volume. Moreover, many ALS patients have anti-calcium channel antibodies capable to provoke apoptotic cell death in vitro through influx of voltage gated calcium channels (Julien, J.-P. (2001) Cell 104, 581-591; Rowland, L. P. & Shneider, N. A. (2001) N. Eng. J. Med. 344, 1688-1699). Thus, blockers of voltage gated calcium might confer benefits. Nimodipine is voltage gated calcium channels blocker that preferentially affects central nervous system (Langley, M. S. & Sorkin, E. M. (1989) Drugs 37, 669-699). Its principal physiological action is to inhibit the influx of extracellular calcium through the voltage-dependent and receptor-operated slow calcium channels in the membranes of myocardial, vascular smooth muscle and neuronal cells.

It would be highly desirable to be provided with a new therapy delaying the onset of the disease and increasing survival time of a subject suffering from a neurodegenerative disease.

### SUMMARY OF THE INVENTION

There is currently no effective pharmacological treatment for amyotrophic lateral sclerosis (ALS). Since there is growing evidence that multiple molecular pathways underlie ALS pathogenesis, it was tested in a mouse model of ALS a combination therapy composed of three generic drugs for distinct targets in the complex pathway to neuronal death. The ALS mice used for drug testing are derived from a well established mouse line, the SODI^{G37R} line 29 with an average life span of above 48 weeks. The cocktail administered in the mouse diet at late stage of disease consisted of minocycline—an antimicrobial agent that inhibits microglial activation, riluzole—a glutamate antagonist, and nimodipine—a voltage gated calcium channel blocker. This combination therapy, called miripine, delayed the onset of disease and increased the average life span of ALS mice by 6 weeks. Remarkably, even when applied after the onset of disease, the miripine therapy increased the longevity of ALS mice by 4 weeks. These results indicate that the miripine three-therapy, which is clinically well tolerated, may represent a novel and effective treatment for ALS having a synergistic effect over the previously discussed three therapy administered alone.

In accordance with the present invention there is provided a method for reducing symptoms related to a neurodegenerative disease and/or treating this neurodegenerative disease, the therapy comprising the administration of a therapeutically effective amount of at least two compounds selected from the group of an inhibitor of microglial activation, an antiglutominergic agent and a voltage gated calcium channer blocker to a patient suffering from the neurodegenerative disease.

The method in accordance with a preferred embodiment of the present invention, wherein the inhibitor of microglial activation is minocycline.

The method in accordance with a preferred embodiment of the present invention, wherein the antiglutaminergic agent is Riluzole.

The method in accordance with a preferred embodiment of the present invention, wherein the voltage gated calcium channel blocker is Nimodipine.

The method in accordance with a preferred embodiment of the present invention, wherein the neurodegenerative disease is selected from the group consisting of: Amyotrophic Lateral Sclerosis (ALS), Alzheimer's disease, Parkinson's disease, Pick's disease, Huntington's chorea, multiple sclerosis, stroke and spinal cord injury.

In accordance with the present invention, there is provided a composition for reducing symptoms related to a neurodegenerative disease and/or treating this neurodegenerative disease, comprising a therapeutically effective amount of at least two compounds selected from the group of an inhibitor of microglial activation, an antiglutaminergic agent and a voltage gated calcium channel blocker in association with a pharmaceutically acceptable carrier.

In accordance with the present invention, there is provided a composition for reducing symptoms related to a neurodegenerative disease and/or treating this neurodegenerative disease, comprising a therapeutically effective amount of at least two compounds selected from the group of an inhibitor of microglial activation, an antiglutaminergic agent and a voltage gated calcium channel blocker, wherein the inhibitor of microglial activation, the antiglutaminergic agent and the voltage gated calcium channel blocker being administered to a subject simultaneously or consecutively.

### BRIEF DESCRIPTION OF THE DRAWINGS

FIG. 1A illustrates Miripine three-therapy increases life span of SOD1^{G37R} mice.

FIG. 1B illustrates Distribution of the mortality of miripine-treated vs control SOD1G37R mice according to their age in weeks;

FIG. 2 illustrates Miripine three-therapy improves muscle strength and delays disease onset; and

FIGS. 3A-3B are histograms showing the total number of axons in L4 and L5 ventral roots of normal mice (WT), drug-treated (tr) and control SOD1^{G37R} mice at the age of 10 months (A) and of 11 months (B);

FIGS. 4A-4) are micrographs showing the immunoreactivities of Cdk5, Cdk4 and activated capsase-3 in the spinal cord of WT mice (A, B, c), of drug-treated SOD1^{G37R} mice (D, E, F) and of control SOD1^{G37R} littermates (G, H, I) at 10 month-old as well as of drug-treated SOD1^{G37R} mice (J, K, L) and of control SOD1^{G37R} littermates (M, N, O) at 11 month-old;

FIGS. 5A-5C are micrographs illustrating the attenuation of microglial activation and astrogliosis in the spinal cord of SOD1^{G37R} mice.

### DETAILED DESCRIPTION OF THE INVENTION

In accordance with the present invention, there is provided a new therapy useful for delaying the onset and increasing the survival time of a subject suffering from a neurodegenerative disease.

Material and Methods

Generation of SOD1^{G37R} Mice

Transgenic mice overexpressing SOD1G37R by ˜5-fold (line 29) (3) were enriched in C57BL/6 background. Only mice heterozygous for the SOD1G37R transgene were used for our study. All mice were genotyped by Southern blotting. The use of animals and all surgical procedures were carried out according to The Guide of Care and Use of Experimental Animals of the Canadian Council of Animal Care.

Three-Therapy Treatment Protocol

The SOD1G37R mice were housed at the standard temperature (21° C.) and in light controlled environment with ad libitum access to the food and water. The study was carried out using transgenic littermates. The mouse littermates were fed a regular rodent food (Harlan, Teklad) and were randomly divided into Three Therapy-treated and control groups, including wild type littermates. At the age of 8-9 months, SOD1G37R mice from the experimental groups were administered triple medicated diet TD 01146 (Harlan, Teklad), containing 1000 mg/kg of minocycline, 500 mg/kg of riluzole and 500 mg/kg of nimodipine. All three compounds were purchased from (Sigma, Oakville,Canada). For the control groups the regular diet was continued until the mice reached end-stage disease. When progression of muscle weakness became marked, mice were fed at the bottom of their cages together with specially designed containers allowing them permanent access to water. Onset of the clinical disease was determined by measurement of motor strength, as described bellow and by the hind limb contraction when mice are suspended by their tail. At the end-stage disease, mice were monitored daily. They were killed when they started to lie on the side in their cages and when they start to express difficulties in grooming. To confirm the effects of combined three-therapy, two independent experiments were carried out on the different sets of trangenic SOD1G37R mice littermates. The therapy was applied at the late presymptomatic stage (7 and 8 months old mice) of disease.

Muscle Strength Test

The mice were allowed to grab vertically oriented wire (˜2 mm in diameter) with the loop at the lower end. The wire was designed in such a way that it allowed the mice to use both fore- and hind limbs. For more consistent measurements, the wire was maintained in the vertically oriented circular motion (circle radius ˜15 cm at 35 r.p.m.). Three tests (three consecutive days) were first used as a learning period trial in which all the mice learned to use both fore and hind limbs in order to stay longer on the circulating wire. Therefore, both skeletal muscle groups contribute in this strength assay. The maximum performance time was cut to 3 min. After the learning period, the test was performed once a week.

Immunohistochemistry

Mice were sacrified by intraperitoneal (I.p.) injection of chlorale hydrate, perfused with 16 g/l sodium cacodylate buffer (pH 7.4) followed by fixative (3% glutoraldehyde in sodium cacodylate buffer). Immunohistochemical studies were performed as previously described. Incubation with the primary antibodies anti-Cdk5 (C-8, 1:1000, Santa Cruz Biotechnology, Santa Cruz, Calif.), anti-glial fibrillary acidic protein monoclonal antibody (anti-GFAP, Sigma, Oakville, Canada, 1:200 dilution), anti-mouse-Mac2 rat monoclonal antibody (TIB-166) distributed by ATCC (Manassas, Va., 1:500 dilution), and anti p-p38, and anti cleaved caspase-3 rabbit polyclonal antibody (New England Biolab, Mississauga, Canada, 1:500 dilution) was performed overnight at room temperature in PBS/BSA. The labeling was developed using a vector ABC kit (Vector Laboratories, Burlington, Canada) and Sigma-fast tablets (Sigma, Oakville, Canada). Tissue section for the axonal counting were prepared for embedding in Epon as described previously.

Western Blots

The mice were sacrificed by overdose of chloralhydrate (i.p.). Immediately after, total protein extracts were obtained from L4-L5 spinal cord sections by homogenization in SDS-urea (0.5% SDS, 8M urea in 7.4 phosphate buffer) with a cocktail of protease inhibitors (PMSF 2 mM, Leupeptine 2 mg/ml, Pepstatin 1 mg/ml and Aprotinin). The protein was measured using a DC-protein assay™ (BioRad, Hercules, Calif.). The proteins were separated on 10% SDS-PAGE, transferred to nitrocellulose membranes and detected using monoclonal primary antibodies against anti-glial fibrillary acidic protein monoclonal antibody (anti-GFAP, Sigma, Oakville, Canada, 1:2000) anti-actin (C-4; 1:5000 Boehringer, Manheim) and anti p-p38, rabbit polyclonal antibody (anti p-p38, Thr 180/Tyr 182, New England Biolab, Mississauga, Canada 1:500 dilution). The Western blots were revealed using the Renaissance chemiluminescence kit (NEN Life Science, Boston, Mass.).

Data Analysis

Data are expressed as a mean±standard error. Statistical significance was assessed by two-tailed student t test (p<0.05).

Results

Miripine Three-Therapy increases the life span of SOD1^{G37R} mice

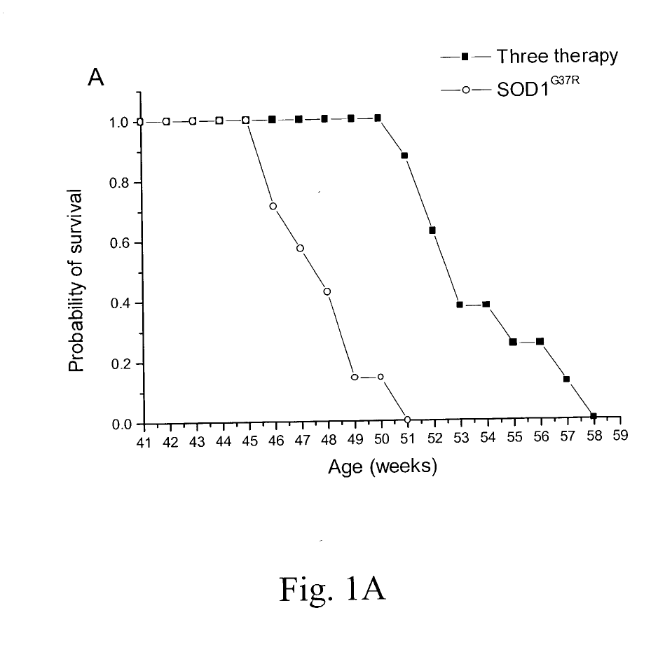

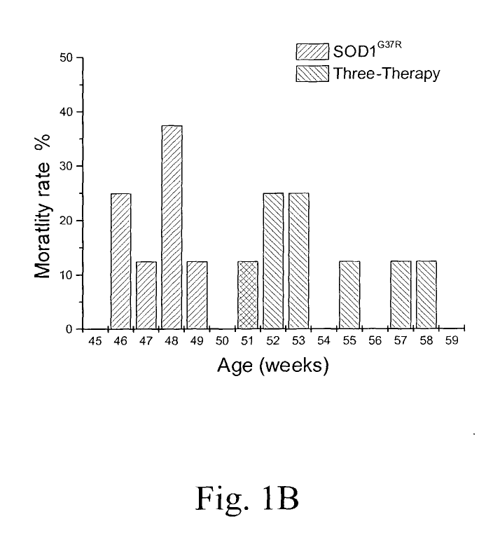

Mouse littermates heterozygous for the SOD1G37R transgene (line 29) were fed a regular rodent food (Harlan, Teklad). At late presymptomatic stage (8 and 9 months), the mice littermates were randomly divided into three-therapy treated and control groups. The three drugs (miripine) were delivered as a dietary supplement in the Special Custom Made Rodent Diet. FIG. 1A shows the survival curves of the miripine-treated (group A) and control SOD1G37R transgenic mice fed on regular diet. In FIG. 1A, the survival probability of transgenic mice is plotted as a function of their age in weeks. It shows that treatment with miripine starting at late presymptomatic stage of disease increased the average life span of SOD^{1G37R} mice by ˜6 weeks. When applied at the late presymtomatic stage, the miripine three-therapy increased longevity of SOD^{1G37R} mice by 7 weeks. As compared to the non-treated littermates, the average life span of miripine-treated SOD1^{G37R} mice was increased by 6 weeks (54.1±0.9; n=10 vs 48.0±0.6; n=10) (Table 1). Remarkably, even when applied after the onset of paralysis, in one group of the animals, the miripine three-therapy slowed down the progression of disease and delayed mortality by 4 weeks.

TABLE 1
Three-therapy delays the onset of disease and increase longevity of
SOD1^{G37R} mice

| | Muscle | Onset of | End Stage |
|---|---|---|---|
| SOD1^{G37R} | Weakness | Paralysis | Paralysis |
| mice | (weeks) | (weeks) | (weeks) |
| Three-Therapy | 47.8 ± 0.95* | 49.6 ± 1.06* | 54.1 ± 0.98* |
| Control | 43.0 ± 0.92 | 45.4 ± 0.59 | 48.0 ± 0.62 |

Values (Mean ± SEM), represents age of the mice expressed in weeks at the time of onset of the different stages of disease.
*significantly different from control (p ≦ 0.05). For both groups n = 10.

As shown in the FIG. 1B, the distribution of mortality rate for the tested mice revealed almost no overlapping between the two tested groups. For non-treated SOD^{1G37R} mice the peak of mortality was at 47-48 weeks, while the morality rate for the treated mice showed more equally spread distribution between 52 and 58 weeks. The difference in life span for some of the miripine-treated vs non-treated animals was more than 10 weeks (FIG. 1B).

Miripine Three-therapy Delays the Onset of Disease and Muscle Strength Decline in SOD1^{G37R} Mice

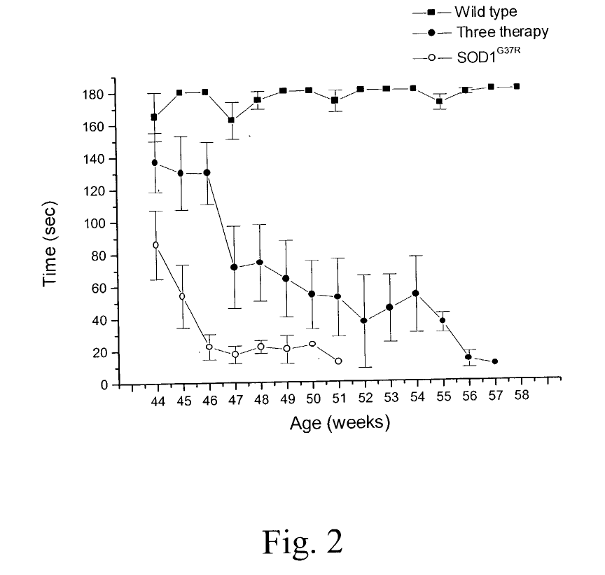

To determine the effects of miripine three-therapy on disease onset and progression in SOD1^{G37R} mice, a muscle strength assay was conducted (see Material and Methods). This assay is based on the time that single mouse was able to grip a vertical circulating wire. The results of the test revealed several different aspects of muscle strength changes associated with different stages of the disease progression. Unlike normal mice, the treated and control SOD1^{G37R} mice showed an age-dependent decline in hanging time (FIG. 2). In FIG. 2, unlike normal mouse littermates, measurement of muscle strength revealed an age-dependent decline in motor performance of SOD1^{G37R} mice. Treatment with miripine three-therapy prevented the decline in muscle strength and significantly improved motor performance of SOD1^{G37R} littermates until end-stage of disease. Muscle strength was indirectly measured as time that mice were able to send hanging on the circulating wire. Each point represents mean±SEM, * significant difference in comparison of miripine-treated vs non-treated SOD1^{G37R} mice, (p≦0.05 by two-tailed t test). The number of animals in each groups were for wild type, n=6; miripine-treated SOD1^{G37R} mice, n=8; control SOD1^{G37R} mice, n=8. The onset of disease in SOD1^{G37R} mice was characterized by a rapid decline in muscle strength (at the age of 43 to 44 weeks), followed by a slower declining stage of muscle strength (46 to 47 weeks of age) progressing to a stage of complete hind limb paralysis. Treatment with miripine three-therapy significantly delayed the first appearance of muscle weakness and significantly improved the motor performance of the treated SOD1^{G37R} mice throughout the tested period (FIG. 2). Applied three-therapy also significantly delayed the onset and slowed down the progression of the disease (FIG. 2 and Table 1).

Effective Protection Against the Loss of Motor Aaxons in SOD1^{G37R} Mice

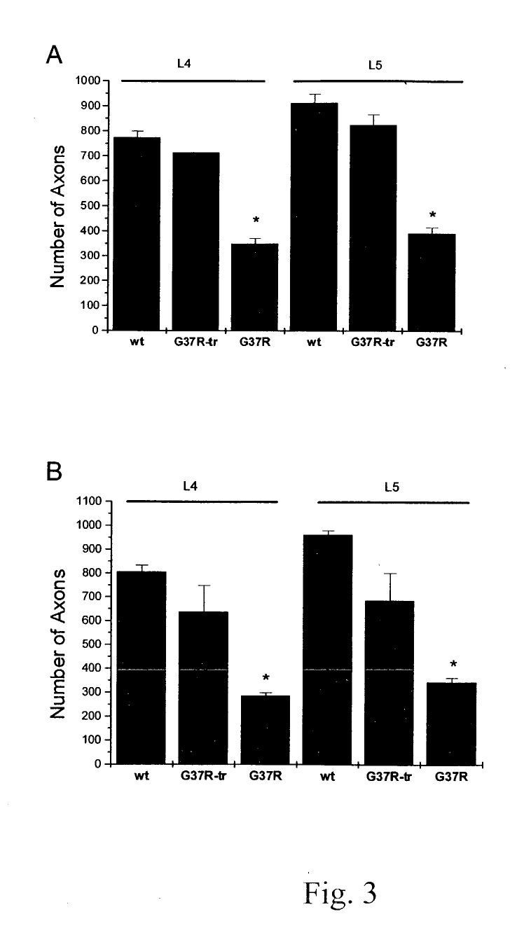

To assess whether the three-drug therapy delayed degeneration of motor neurons, the total number of motor axons in L4 and L5 ventral roots from treated SOD1^{G37R} mice (n=3 or 4) and control SOD1^{G37R} littermates (n=4) at early symptomatic stage of disease (44 weeks) and at late stage of disease (48 weeks) was counted as shown in FIGS. 3A and 3B. At early stage of disease, motor axons from treated SOD1G37R mice were mostly spared unlike axons from control SOD1^{G37R} littermates (FIG. 3A). For instance, at 44 week-old, the L5 ventral roots from control SOD1^{G37R} mice had 390±23 remaining axons whereas those from drug-treated mice had 823±41 axons, which is not significantly different from control values (911±36). A similar pattern was observed at the level of L4 ventral roots (713±20 for drug-treated vs. 352±20 for control SOD1^{G37R} mice). At 48 week-old, the number of remaining axons in the L4 and L5 ventral roots from drug-treated SOD1^{G37R} mice were of 637±112 and 685±115, respectively. Thus, while some axonal loss was evident at 48 weeks, the majority of motor axons were still present in the drug-treated SOD1^{G37R} mice.

Reduced Cdk5 Mislocalization and Capsasa-3 Activation

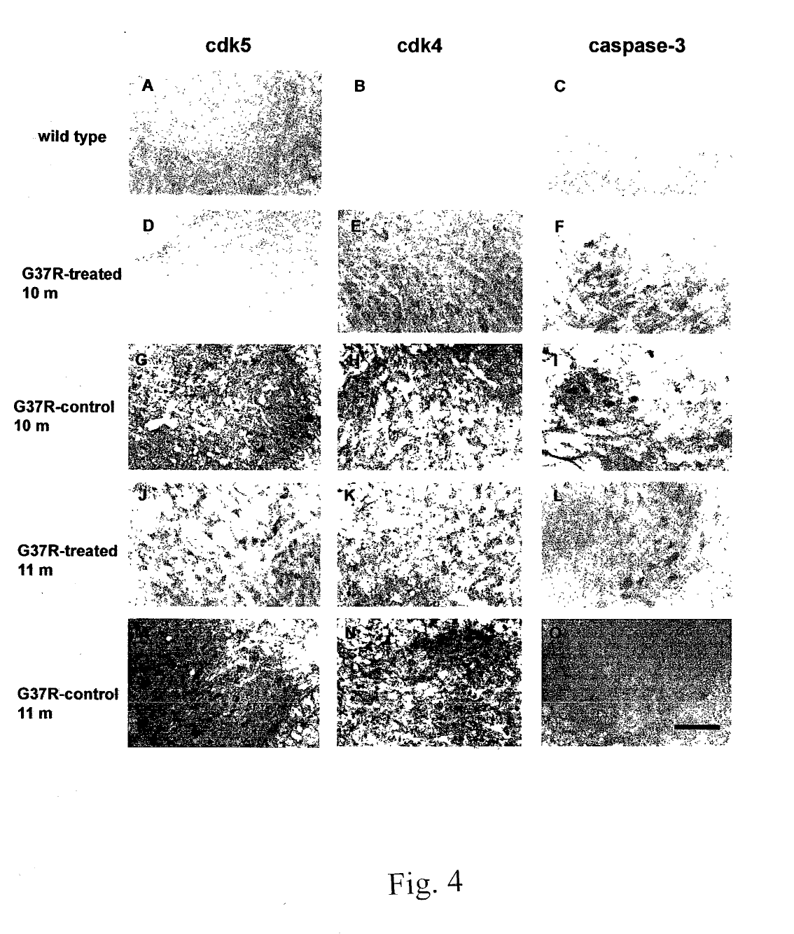

Recent studies demonstrated the involvement of caspase-3 activation in ALS pathogenesis. Activation of caspase-3 occurs late in the course of disease and it is associated with the loss of large motor neurons. Two other pathological hallmarks of degenerating neurons in SOD1^{G37R} mice are the mislocalization of Cdk5³⁰ and the nuclear localization of Cdk4³¹. Cdk5 is normally targeted to the cell membrane by its activator p35. However, in SOD1^{G37R} mice, Cdk5 is mostly detected in the cytoplasm of motor neurons. To examine whether the three-drug therapy attenuated the signals for markets of neurodegeneration in the spinal cord sections of SOD1^{G37R} mice, immunohistochemistry with anti-Cdk5, anti-Cdk4 and anti-caspase-3 antibodies were carried out.

Whereas the spinal motor neurons of control SOD1^{G37R} mice exhibited robust immunoreactivities for Cdk5 and Cdk4 at 10 month-old (FIGS. 4C and 4H), very low immunoreactivities were detected for Cdk5 and Cdk4 in spinal cord sections of 10 month-old SOD1^{G37R} mice under drug treatment (FIGS. 4D and 4E). At the age of 11 months, immunoreactivities for Cdk5 and Cdk4 were detected in spinal motor neurons of drug-treated SOD1^{G37R} mice but at reduced levels as compared to control SOD1^{G37R} mice (FIGS. 4J, 4K, 4M and 4N).

Antibodies against activated form of caspase-3 yielded a weak cytoplasmic immunostaining in several spinal motor neurons of drug-treated SOD1^{G37R} mice at 10 month-old (FIG. 4F), indicating that caspase-3 activation preceded axonal degeneration. Again, much stronger caspase-3 immunoreactivity was detected in moto neurons of 10 month-old control SOD1^{G37R} littermates. This shows that the beneficial effects of the three-drug treatment are associated with reduced signals for markers of neurodegeneration.

Three-drug Therapy Attenuates Astrocytosis and Microglial Activation

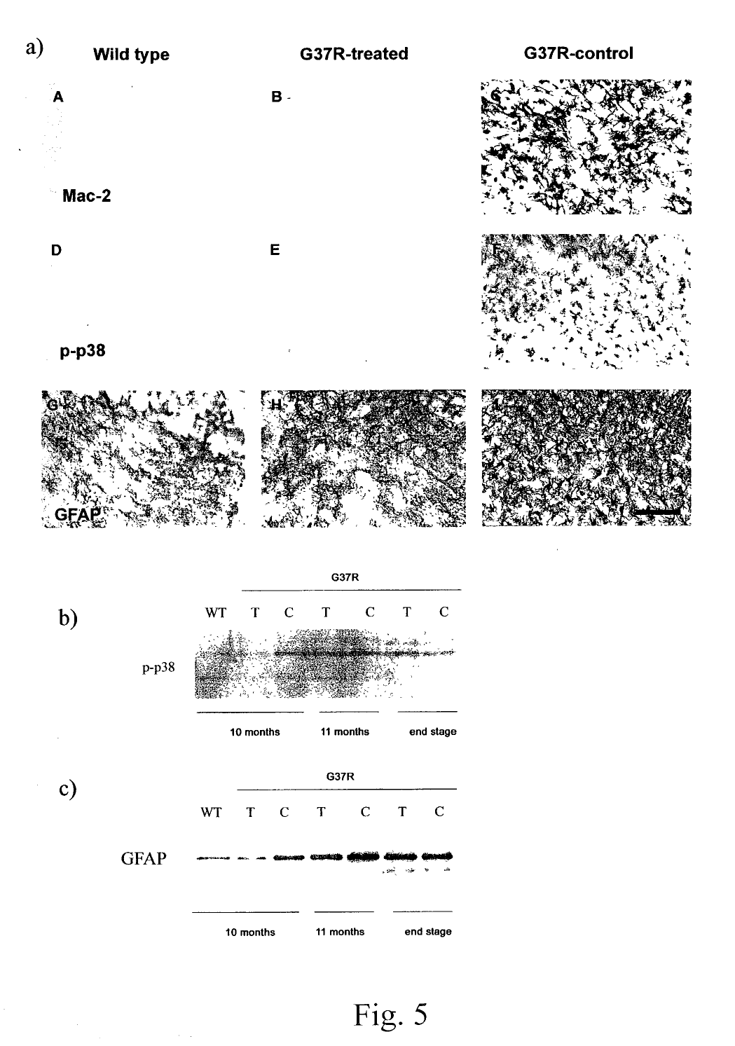

Astrocytosis and microgliosis are non-neuronal events that are likely to contribute to the neurodegenerative processes in ALS. Recently, it was shown that minocycline alone attenuates microglial activation but not astrocytosis in SOD1^{G37R} mice. To determine whether inclusion of nimodipine and riluzole together with minocycline exerted additional effects on glial cell activation in SOD1^{G37R}, immunohistochemistry and western blotting expression of Mac-2 and phosphorylated form p38 MAPK (p-p38) which are markers of microglial activation, and GFAP as a marker of astrogliosis were examined. At early symptomatic stage (44 weeks), the spinal cord sections of age-matched normal mice and drug-related SOD1^{G37R} mice were almost completely devoid of Mac-2 immunoreactivity (FIG. 5). In contrast, the spinal cord of control SOD1^{G37R} mice showed a robust Mac-2 immunoreactivity. The Mac-2 immunoreactive cells revealed morphology typical of activated microglia/macrophages (irregular shape, short processes) (FIG. 5A, panel C). A similar pattern of immunoreactivity was observed with antibodies against p-p38. Control SOD1^{G37R} mice yielded a strong p-p38 immunoreactivity in the white and gray matter (predominantly ventral horns) of the spinal cord (FIG. 5A, panel F). The p-p38 signal was considerably attenuated by the three-drug treatment. At the age of 44 weeks, the p-p38 immunoreactivity in the spinal cord of drug-treated SOD1^{G37R} mice was a low as in normal mice (FIG. 5a, panels D and E). this was further confirmed by western blotting. At age of 10 months weeks, spinal cord extracts from normal mice or from drug-treated SOD1^{G37R} mice (FIG. 5B). During disease progression, the levels of p-p38 in spinal cord extracts gradually increased in drug-treated SOD1G37R mice.

Unlike minocycline alone, the presence of riluzole and nimodipine in the three-drug therapy of the present invention markedly attenuated GFAP immunoreactivity in spinal cord sections of SOD1^{G37R} mice at 44 week-old (FIG. 5A, panels G and H). This was further confirmed by the weak GFAP immunostaining on western blot of spinal cord extracts from drug-treated SOD1^{G37R} mice as compared to control SOD1^{G37R} littermates (FIG. 5C).

Discussion

It is reported here for the first time a three-therapy pharmacological approach (combination of minocycline, riluzole and nimodipine) which is effective in delaying the onset of disease and mortality in a mouse model of ALS. Starting at the late presymptomatic stage (8 or 9 months of age) administration of miripine three-therapy in the diet significantly delayed the onset of motor neuron degeneration, attenuated astrogliosis and microglial activation, slowed down the disease progression and increased the motor performance of SOD1^{G37R} mice. This three-therapy approach delayed the onset of disease and increased the average longevity of ALS mice by 6 weeks. Moreover, for some mice the increase in life span exceeded 10 weeks.

Minocycline is a semisynthetic tetracycline derivative that effectively crosses blood-brain barrier and it is extensively used in human with relatively little side effects. It has been suggested that minocycline exerts neuroprotective effects by preventing microglial activation and reducing the induction of caspase-1, thereby decreasing the level of mature proinflammatory cytokine IL-1β (Yrjänheikki, J., et al. (1998) Proc. Natl. Acad. Sci. USA 95, 15769-15774; Yrjänheikki, J., et al. (1999) Proc. Natl. Acad. Sci. USA 96, 13496-13500; Chen, M., et al. (2000) Nat. Med. 6, 797-801). In addition, it has been shown that minocycline, doxycycline and their non-antibiotic derivatives (chemically modified tetracyclines) inhibit matrix metalloproteases, nitric oxide synthases, protein tyrosin nitration, cyclooxygenase-2 and prostaglandine E2 production. Recent studies performed with primary neurons and purified microglial cultures demonstrated that minocycline may also confer neuroprotection through inhibition of excitotoxin-induced microglial activation. Minocycline inhibits glutamate- and kainate-induced activation of p38 MAPK, exclusively activated in microglia.

A protection mechanism based on attenuation of microglial activation is compatible with an inflammation involvement in the pathology of neurodegenerative disorders. In human ALS, reactive microglia and reactive astrocytes are abundant in affected areas. Such gliosis as a phenomenon occurs also in the SOD1^{G37R} mouse model described here. It is known that minocycline, as a single therapy, slowed down progression of disease in SOD1^{G37R} mice but without affecting the onset. This shows that activated microglia, through the release of pro-inflammatory molecules, are more likely to play an active role in later stages of disease, contributing more to spreading of the neurodegenerative process (Julien, J.-P. (2001) Cell 104, 581-591).

Riluzole, a glutamate antagonist, is the only drug currently approved for therapy of ALS with only marginal effects on survival (Rowland, L. P. & Shneider, N. A. (2001) N. Eng. J. Med. 344, 1688-1699). In two controlled clinical trials it increased survival of ALS patients by 3-6 months. Although the precise mechanism of action of riluzole has not been fully elucidated, it appears to involve interference with excitatory amino acid (EAA) in the CNS, possibly through inhibition of glutamic acid release, blockade or inactivation of sodium channels and/or activation of G-protein coupled transduction pathways. When tested as a single therapy in SOD1 mutant mice it increased survival for 13-15 days without affecting the onset of disease (Gurney, M. E., et al. (1996) Ann. Neurol. 39, 147-157).

At the present, there is enough substantial evidence supporting hypothesis that Ca2+ influx through L-type voltage gated channels may contribute to neuronal death. Recent study by demonstrated that Ca2+ entry through L-type calcium channels induces mitochondrial disruption and cell death. In addition, antibodies against voltage gated calcium channels have been isolated from cerebrospinal liquor of some ALS patients, and when tested in in vitro and in vivo condition they induced selective increase of intracellular calcium in motor neurons associated with cell injury and death. Calcium channel blocking agents antagonize EAA receptor and decrease calcium entry into damaged neurons that may slow down or reverse neurodegenerative processes in ALS.

Nimodipine is the L-type voltage gated calcium channel blocker with preferential effects on CNS (Langley, M. S. & Sorkin, E. M. (1989) Drugs 37, 669-699). It exerts anxiolytic and antiamnestic effect in animals, it facilitates learning in old animals, exhibits certain neuroprotective effects in ischemia/hypoxia induced nerve damage, posses some anticonvulsant properties. Recently, it has been shown that nimodipine promotes regeneration and functional recovery after intracranial facial nerve crush. However, tested as a mono-therapy in one controlled clinical trial nimodipine was not effective in slowing down the disease progression in ALS patients.

Previous studies indicated that nimodipine and riluzole when applied in human ALS or mouse model as a single therapy exert very modest or no effects on ALS. In contrast, minocycline was quite effective in slowing down the disease progression in mouse model of ALS. However, combination of minocyline, riluzole and nimodipine, applied as a three-therapy increased average life span of SOD1G37R mice by 6 weeks (FIG. 1, Table 1), which represents 100% increase in efficacy as compared to minocycline therapy. Remarkably, for some of the animals difference in the life span was more than 10 weeks (see FIG. 2). As shown in the FIG. 3 and Table 1, treatment with three-therapy also significantly delayed decline of the muscle strength and disease onset of SOD1G37R mice.

To date, aside from miripine three-therapy, no pharmacological treatment was able to delay both, the onset and the progression of disease in a mouse model of ALS. Comparing the effectiveness of our three-therapy approach to a pharmacological efficacy of every single compound contained in our drug cocktail, it is evident that they acted in synergy. Multiple factors and pathological pathways, that are not mutually exclusive, are involved in the pathogenesis of ALS and the disease progression. Our results clearly demonstrated that strategic and simultaneous pharmacological intervention on three different pathological pathways, riluzole as antiglutaminergic agent prevents excitotoxic effects of glutamate; nimodipine as voltage gated calcium channel blocker prevents excessive calcium influx into depolarized damaged neurons and minocycline, as a inhibitor of microglial activation, prevents toxic effects of activated microglia, resulted in remarkably effective treatment.

The miripine three-therapy is also suitable to be used for the reduction of symptoms and/or treatment of other related neurodegenerative diseases like Alzheimer's disease, Pick's disease, Parkinson's disease, multiple sclerosis and Huntington's chorea. It may also be useful for the treatment of spinal cord injuries and stroke.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for reducing symptoms related to a neurodegenerative disease and/or treating said neurodegenerative disease, said therapy comprising the administration of a therapeutically effective amount of at least two compounds selected from the group of an inhibitor of microglial activation, an antiglutominergic agent and a voltage gated calcium channer blocker to a patient suffering from said neurodegenerative disease.

2. The method of claim 1, wherein said inhibitor of microglial activation is minocycline.

3. The method of claim 1, wherein said antiglutaminergic agent is Riluzole.

4. The method of claim 1, wherein said voltage gated calcium channel blocker is Nimodipine.

5. The method of claim 1, wherein said neurodegenerative disease is selected from the group consisting of: Amyotrophic Lateral Sclerosis (ALS), Alzheimer's disease, Parkinson's disease, Pick's disease, Huntington's chorea, multiple sclerosis, stroke and spinal cord injury.

6. The method of claim 1, wherein said neurodegenerative disease is ALS.

7. The method of claim 1, wherein said compounds are administered simultaneously.

8. The method of claim 1, wherein said compounds are administered consecutively.

9. A composition for reducing symptoms related to a neurodegenerative disease and/or treating said neurodegenerative disease, comprising a therapeutically effective amount of at least two compounds selected from the group of an inhibitor of microglial activation, an antiglutaminergic agent and a voltage gated calcium channel blocker in association with a pharmaceutically acceptable carrier.

10. The composition of claim 9, wherein said inhibitor of microglial activation is minocycline.

11. The composition of claim 9, wherein said antiglutaminergic agent is Riluzole.

12. The composition of claim 9, wherein said voltage gated calcium channel blocker is Nimodipine.

13. The composition of claim 9, wherein said neurodegenerative disease is selected from the group consisting of: Amyotrophic Lateral Sclerosis (ALS), Alzheimer's disease, Parkinson's disease, Pick's disease, Huntington's chorea, multiple sclerosis, stroke and spinal cord injury.

14. The composition of claim 9, wherein said neurodegenerative disease is ALS.

15. A composition for reducing symptoms related to a neurodegenerative disease and/or treating said neurodegenerative disease, comprising a therapeutically effective amount of at least two compounds selected from the group of an inhibitor of microglial activation, an antiglutaminergic agent and a voltage gated calcium channel blocker, wherein said inhibitor of microglial activation, said antiglutaminergic agent and said voltage gated calcium channel blocker being administered in association with a pharmaceutically acceptable carrier to a subject simultaneously or consecutively.

16. The composition of claim 15, wherein said inhibitor of microglial activation is minocycline.

17. The composition of claim 15, wherein said antiglutaminergic agent is Riluzole.

18. The composition of claim 15, wherein said voltage gated calcium channel blocker is Nimodipine.

19. The composition of claim 15, wherein said neurodegenerative disease is selected from the group consisting of: Amyotrophic Lateral Sclerosis (ALS), Alzheimer's disease, Parkinson's disease, Pick's disease, Huntington's chorea, multiple sclerosis, stroke and spinal cord injury.

20. The composition of claim 15, wherein said neurodegenerative disease is ALS.
